(19) United States
(12) Patent Application Publication
(10) Pub. No.: US 2025/0276009 A1
(43) Pub. Date: Sep. 4, 2025

(54) Therapeutic use of hydrogen sulfide in frostbite injury

(71) Applicant: [(The)] London Health Sciences Centre Research Inc., London (CA)

(72) Inventors: Alp SENER, London (CA); George Johnson DUGBARTEY, London (CA)

(73) Assignee: [(The)] London Health Sciences Centre Research Inc., London (CA)

(21) Appl. No.: 19/069,453

(22) Filed: Mar. 4, 2025

Related U.S. Application Data
(60) Provisional application No. 63/561,201, filed on Mar. 4, 2024.

(51) Int. Cl. A61K 33/04; A61K 9/06; A61K 31/67; A61K 31/675; A61K 31/685; A61K 31/728; A61P 17/02

(52) U.S. Cl. CPC A61K 33/04; A61K 9/06; A61K 31/67; A61K 31/675; A61K 31/685; A61K 31/728; A61P 17/02

(57) ABSTRACT

A method of treating frostbite injury in a patient comprising administering to the patient H₂S. Also, a composition comprising H₂S and a skin moisturizer.

### CROSS REFERENCE TO RELATED APPLICATIONS

This application claims the benefit under 35 U.S.C. 119(e) of U.S. Provisional Ser. No. 63/561,201, filed Mar. 4, 2024, the contents of each of which are hereby incorporated by reference into the present disclosure.

### FIELD OF TECHNOLOGY

The present disclosure relates to the therapeutic use of hydrogen sulfide in frostbite injury.

### BACKGROUND INFORMATION

Frostbite is a thermal injury caused when skin and/or tissue are exposed to sub-zero temperatures (in degrees Celsius). Frostbite typically affects the peripheral areas of the body and is associated with limb loss and high rates of morbidity. Historically, treatment options have been limited to supportive care, leading to suboptimal outcomes for affected patients. There is no effective treatment for frostbite apart from supportive care, such as rewarming of tissue, daily wound care and treatment with analgesics.

Frostbite injury differs from that of other skin ailments such as abrasions and cuts in that its pathophysiology relies on a significant reduction in blood flow through severe vasoconstriction and rapid cooling of tissues leading to crystallization and ice formation, cellular and endothelial injury, microvascular injury and eventual cellular death and thrombosis. During rewarming, there is a significant reperfusion injury due to the release of various cytokines, chemokines and inflammatory mediators.

Therefore, there is a need to identify novel pharmacological agents for effective treatment of frostbite injury.

### SUMMARY OF DISCLOSURE

In one embodiment, the present disclosure relates to a method of treating frostbite injury in a patient comprising administering to the patient H₂S.

In another embodiment, the present disclosure provides for a method of preventing frostbite injury in a subject comprising administering to the subject H₂S.

In one embodiment of the methods of the present disclosure, the administering comprises topically contacting the frostbite injury with the H₂S.

In another embodiment of the methods of the present disclosure, the H₂S is provided as a H₂S donor.

In another embodiment of the methods of the present disclosure, the H₂S is provided in a composition comprising the H₂S and a vehicle.

In one embodiment of the methods of the present disclosure, the vehicle contains hyaluronic acid.

In another embodiment of the methods of the present disclosure, the vehicle contains phosphatidylethanolamine.

In another embodiment of the methods of the present disclosure, the vehicle contains phosphatidylethanolamine and hyaluronic acid.

In another embodiment of the methods of the present disclosure, the vehicle is a water-in-oil emulsion.

In another embodiment of the methods of the present disclosure, the vehicle is an oil-in-water emulsion.

In another embodiment of the methods of the present disclosure, the vehicle is a commercially available skin moisturizer.

In another embodiment of the methods of the present disclosure, the H₂S donor is selected from STS, AP39 and GYY4137.

In another embodiment, the present disclosure relates to a composition comprising H₂S and a skin moisturizer.

In one embodiment of the composition of the present disclosure, the H₂S is provided as a H₂S donor.

In another embodiment of the composition of the present disclosure, the H₂S donor is selected from STS, AP39 and GYY4137.

In another embodiment of the composition of the present disclosure, the skin moisturizer comprises hyaluronic acid and/or phosphatidylethanolamine.

In another embodiment of the composition of the present disclosure, the skin moisturizer is a commercially available skin moisturizer.

### BRIEF DESCRIPTION OF THE DRAWINGS

The following figures illustrate various aspects and preferred and alternative embodiments.

FIGS. 1A to 1F: FIGS. 1A to 1E are photographs illustrating animal preparation. 1A: hair covering the required dorsal area of the back of the animal is completely removed (shaved). 1B: A circle of 2.9 cm in diameter is drawn on the shaved dorsal area the animal's back. 1C: The circle is drawn using a permanent marker. 1D: Frozen magnets are placed on opposite sides of skinfold within the marked circle. 1E: Skin is shown immediately after thawing. 1F: Flowchart of the experiment design for induction of frostbite.

FIG. 2. Progression of frostbite wound (injury) from day 4 to day 7 to day 15 after induction of frostbite injury for animals treated with vehicle control (cream containing hyaluronan and phosphatidylethanolamine), 200 nm AP39 in vehicle control, 1 μm AP39 in vehicle control, 150 μm STS in vehicle control and 200 μmol/kg GYY4137 in vehicle control.

FIGS. 3A to 3B. Graphs illustrating quantification of the wound areas of FIG. 2 at day 4 after induction of frostbite injury (FIG. 3A) and at day 15 after induction of frostbite injury (FIG. 3B). [*/**/***/****p<0.005/0.01/0.001/0.0001]

FIG. 4. Photographs showing progression of frostbite wound (injury) from day 4 to day 7 to day 15 after induction of frostbite injury for animals treated with 4 different types of vehicle controls (Aveeno®, Dove®, Neutrogena® and Nivea®) and treated with 150 μm STS in each of the 4 vehicles.

FIGS. 5A to 5B. Graphs illustrating quantification of the wound areas of FIG. 4 at day 4 after induction of frostbite injury (FIG. 5A) and at day 15 after induction of frostbite injury (FIG. 5B). [*/**/***p<0.05/0.001/0.001]

FIGS. 6A to 6I. 6A to 6G: Microphotographs of histopathological changes on day 15 following induction of frostbite injury of healthy controls (6A) and crush controls (6B) and 15 days following induction of frostbite injury and treatment with vehicle control (6C), 200 nm AP39 (6D), 1 μm AP39 (6E), 200 μmol/kg GYY4137 (6F) and 150 μm STS (6G). 6H: Graph illustrating quantification of acute and chronic inflammation, granulation tissue formation, fibroblast maturation and re-epithelialization for (from left to right of each group) healthy control (first bar from the left), crush control (second bar from the left), vehicle control (third bar from the left), 200 nm AP39 (fourth bar from the left) and 1 μm AP39 (fifth bar from the left). 4I: Graph illustrating quantification of acute and chronic inflammation, granulation tissue formation, fibroblast maturation and re-epithelialization for (from left to right of each group) 200 μmol/kg GYY4137 (first bar from the left) and 150 μm STS (second bar from the left). For FIGS. 3H and 3I: *p<0.05 vs. vehicle control, **p<0.01 vs. vehicle control, ***p<0.001 vs. vehicle control.

FIG. 7. Microphotographs showing immunofluorescence staining of markers of wound healing (from top row to bottom row: cleaved caspase-3, CD31, KI-67, CD163, fibronectin and cytokeratin) for (from left column to right column): healthy control, crush control, vehicle control, 200 nm AP39, 1 μm AP39, 150 μm STS and 200 μmol/kg GYY4137.

FIGS. 8A to 8F. Quantification of immunofluorescence staining indicating the degree of apoptosis (8A), vascularization and angiogenesis (8B), fibroblast cell proliferation (8C), anti-inflammatory activity (8D), granulation tissue formation (8E), and keratinocyte proliferation (8F). *p<0.05 vs. vehicle control, ***p<0.01 vs. vehicle control, ****p<0.001 vs. vehicle control. For each of 8A to 8F, from left to right the bars are healthy control, crush control, vehicle control, 200 nm AP39, 1 μm AP39, 200 μmol/kg GYY4137 and 150 μm STS.

FIG. 9. Graphs showing quantification of immunofluorescence staining indicating caspase-3 expression at day 15 after induction of frostbite injury. **/***p<0.01.0001; n=5.

FIG. 10. Graphs showing quantification of immunofluorescence staining indicating CD31 expression at day 15 after induction of frostbite injury. **/***p<0.01.0001; n=5.

FIG. 11. Graphs showing quantification of immunofluorescence staining indicating KI-67 expression at day 15 after induction of frostbite injury. **/***p<0.01.0001; n=5.

FIG. 12. Graphs showing quantification of immunofluorescence staining indicating CD163 expression at day 15 after induction of frostbite injury. */**p<0.05/0.01; n=5.

FIG. 13. Graphs showing quantification of immunofluorescence staining indicating fibronectin expression at day 15 after induction of frostbite injury. */**p<0.05/0.01; n=5.

FIG. 14. Graphs showing quantification of immunofluorescence staining indicating cytokeratin expression at day 15 after induction of frostbite injury. **p<0.01; n=5.

### DETAILED DISCLOSURE

### Definitions

In this specification and in the claims that follow, reference will be made to several terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

The term “about,” particularly in reference to a given quantity, is meant to encompass deviations of plus or minus five percent.

“Administration” can be carried out in one dose, continuously or intermittently throughout the course of treatment. Methods of determining the most effective means and dosage of administration are known to those of skill in the art and will vary with the composition used for therapy, the purpose of the therapy, the target cell being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, nasal administration, injection and topical application.

“Prevent” is used to mean obtaining a prophylactic effect in terms of completely or partially preventing frostbite injury from occurring.

A “subject” is an animal susceptible to frostbite injury, including mammals or a human. A patient is a subject in need of treatment.

As used herein, the terms “treating,” “treatment” and the like are used herein to mean a partial or complete cure frostbite injury and/or adverse effect attributable to frostbite injury.

### Overview

The present disclosure relates to methods of treating and/or preventing frostbite injury with hydrogen sulfide and with composition comprising hydrogen sulfide. In embodiments, the present disclosure relates to hydrogen sulfide for the treatment and/or prevention of frostbite related injuries.

In one embodiment, the present disclosure relates to a method of treating frostbite injury in a patient comprising, or alternatively consisting of, or alternatively consisting essentially of, administering to the patient H₂S. In one embodiment, the method of treating frostbite injury in a patient comprises, or alternatively consists of, or alternatively consists essentially of, topically contacting the frostbite injury with H₂S.

In another embodiment, the present disclosure relates to a method of preventing frostbite injury in a subject comprising, or alternatively consisting of, or alternatively consisting essentially of, administering to the subject H₂S.

In another embodiment, the present disclosure relates to a method of preventing frostbite injury in a subject comprising, or alternatively consisting of, or alternatively consisting essentially of, topically contacting the subject's skin with H₂S. In embodiments, the topical contact is local, i.e., at the site of the frostbite injury.

The H₂S can be provided as H₂S or as a H₂S donor. In one embodiment of the disclosure, the H₂S is provided as a H₂S donor. Non-limiting examples of H₂S donors include AP39, GYY4137 and sodium thiosulfate (STS).

The compositions of the present disclosure when applied to a frostbite injury have been shown to improve granulation tissue maturation (FIGS. 8E and 13), angiogenesis (FIGS. 8B, 10 and 12), cell proliferation (FIGS. 8C and 11) and keratinocyte proliferation and differentiation to restore epidermal barrier when compared to vehicle control. Thus, H₂S donor compounds improved frostbite wound healing, and therefore the compositions of the present disclosure comprising H₂S and H₂S donor compounds are useful in the treatment of frostbite injury in a subject or patient.

Typical levels of hydrogen sulfide contemplated for use in accordance with present disclosure include about nM to μM concentrations of slow-releasing H₂S donors. For example, in the case of AP39 typical concentration may be from about 200 nM to about 1 μM, in the case of STS from about 200 nM to about 200 UM and in the case of GYY4137 about 200 μmol/kg of GYY4137. The safety and effective dose of the donor molecules has been established in high mammals or in the context of other human pathologies.

In one embodiment, the H₂S or H₂S donor is provided in a composition comprising the H₂S or H₂S donor in combination with a vehicle base cream or lotion or moisturizer, etc. In this document, the terms “vehicle” or “vehicle base” are used interchangeably to refer to a cream, lotion or moisturizer base, including commercially available creams, lotions or moisturizers, such as skin moisturizers, skin creams, hand cream, foot cream, body cream, lip cream, lip gloss, lip stick, gel, balm and lotion to the skin surface. In one embodiment, the vehicle may be one or more sold under the brand names Aveeno®, Nivea®, Neutrogena® and Dove®.

In one embodiment, the vehicle base contains hyaluronic acid. In one embodiment, the vehicle cream or lotion or moisturizer contains phosphatidylethanolamine. In one embodiment, the vehicle cream/lotion/moisturizer contains hyaluronic acid and phosphatidylethanolamine. In another embodiment, the vehicle cream/lotion/moisturizer is provided as a water-in-oil emulsion. In another embodiment, the vehicle is an oil-in-water emulsion.

In another embodiment, the present disclosure provides for a composition comprising H₂S and skin cream/lotion/moisturizer, including the vehicle base cream/lotions/moisturizers described above. In one embodiment of the composition, the H₂S is provided as a H₂S donor. In another embodiment, the H₂S donor is selected from STS, AP39 and GYY4137.

In order to aid in the understanding and preparation of the present disclosure, the following illustrative, non-limiting examples are provided.

### EXAMPLES

### Example 1

### Ethical Statement and Animal Grouping

The Animal Use Committee of the University of Western Ontario approved all experimental procedures. The experimental procedures were considered as Category D from the Canadian Council of Animal Care's Categories of Invasiveness for Animal Experiments. A total of 25 B57BL/6 mice aged between 8-10 weeks old were obtained from Charles River Canada (Senneville, Quebec, Canada) and housed 5 per cage under a 12-hour light/dark cycle at the Health Sciences Animal Care Facility at the University of Western Ontario (London, Ontario, Canada). Mice were randomly assigned to 5 groups being healthy control (n=5), crush control (n=5), vehicle control (n=5), 200 nM AP39 (n=5) and 1 μM AP39 (n=5).

### Skin Preparation

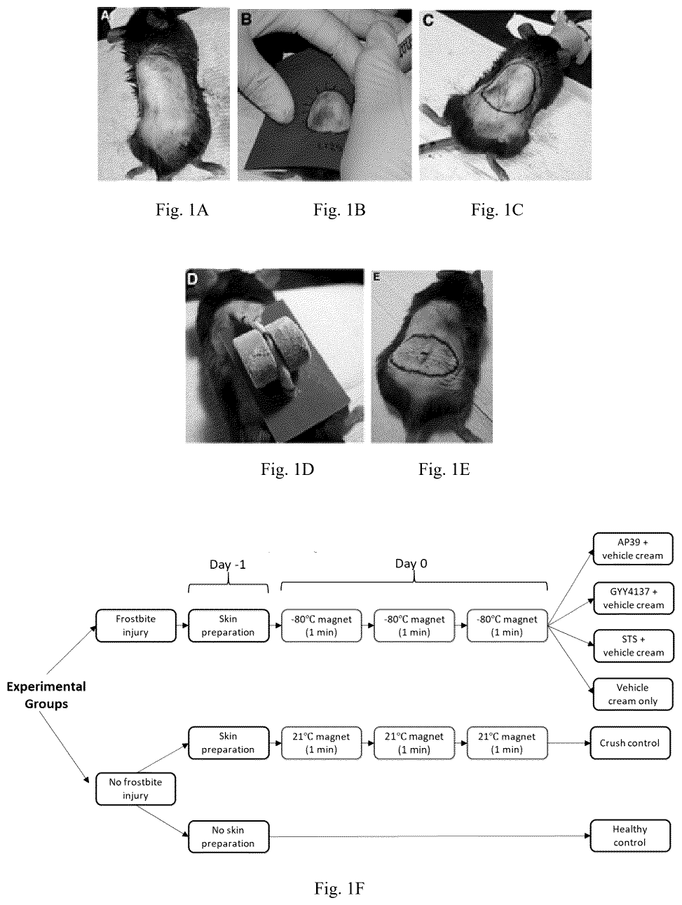

To ease the process of skin preparation, mice were initially anesthetized with a combination of 20 mg/ml xylazine and 100 mg/mL ketamine intraperitoneally, and the dorsal fur of the animals were shaved using electric clippers from the top of the hind legs to the base of the neck. A depilatory cream was then used to remove any fur still remaining. General anesthesia was maintained by inhalation of 5% isoflurane and oxygen delivered at 400 mL/minute to minimize the time of recovery post-anesthesia. Following complete fur removal, the skin of the animal was cleaned completely of the hair removal cream using an alcohol prep pad. Next, a silicone sheet was used as a template to trace a uniform circle on the surface of the skin using a permanent marker. The circular outline on the back of the animal was used as a guide during frostbite injury (see FIGS. 1A to 1E).

### Cream Preparation and Application

Commercially available skin creams (Aveeno®, Dove®, Neutrogena® and Nivea®) were used as vehicle creams to deliver the H₂S donor (AP39, STS and GYY4137) in order to penetrate the stratum corneum (outermost layer of the skin), which acts as a physical barrier. The vehicle cream containing hyaluronan and phosphatidylethanolamine (referred to as HEPE) as active ingredients, was provided by Dr. Eva Turley, and allowed for delivery of AP39 to the vascularized layers of the dermis [2]. AP39 was generously provided by Dr. Matthew Whiteman (Exeter, UK) and diluted to a stock concentration of 1 mM in DMSO. STS was purchased from Seaford Pharmaceuticals Inc., Mississauga, ON, Canada while GYY4137 was purchased from MedChemExpress, NJ, USA. To prepare the cream+H₂S donor compound, the H₂S donor was added via micropipette combined by stirring and subsequent mixing using a vortex device to final concentrations of 200 nM, 1 UM and 150 μM.

### Induction of Frostbite Injury

The protocol of the frostbite model is illustrated in FIG. 1B. Based on a previously validated model [1], 100% ceramic magnets (diameter 18.0 mm, thickness 5.0 mm, weight 4.5 g) were used to induce frostbite injury. Magnets were frozen in dry ice (−80° C.) prior to animal skin preparation. The dorsal skin of C57BL/6 mice (Mus musculus) was pinched to create a skin fold and a silicone sheet was placed underneath as illustrated in FIG. 1D. The silicone sheet acted as a barrier to prevent the mice from possible systemic hypothermia while creating a precise injury and maximizing survival. Two frozen magnets were then placed on the skin from opposite sides of the skin fold such that they adhered with the middle of the circle outline at the peak of the fold's center as described by Auerbach et al., [1] and illustrated in FIG. 1D. After 1 minute, the magnets were removed and new frozen magnets simultaneously placed in order to keep the freezing temperature of the magnets consistent and ensured that no thawing of the skin occurred. A total of 3 magnet exchange cycles occurred in order to induce frostbite injury to the mice for 3 minutes after which the skin was allowed to thaw completely. Core temperature of each mouse was continuously monitored using a rectal thermometer and did not change. The animals were placed on a heating pad set to 37° C. to mitigate the risk of death due to systemic hypothermia. Upon induction of frostbite injury, the mice were randomly assigned to the following groups: vehicle cream control, 200 nM AP39 plus vehicle cream, 1 μM AP39 plus vehicle cream, 150 μM STS plus vehicle cream, 200 μmol/kg GYY4137 plus vehicle cream, and received daily topical application of vehicle cream without H₂S donor, vehicle cream containing 200 nM AP39 (200 μL), vehicle cream containing 1 μM AP39 (200 μL), vehicle cream containing 150 μM STS (200 μL) and vehicle cream containing 200 μmol/kg GYY4137 (200 μL) respectively until time of sacrifice. As AP39 was synthesized in-house [3], we chose these two concentrations (200 nM and 1 μM) due to our previous success with the former concentration in mitigating cold IRI [4] while the latter concentration was based upon our work in vitro which suggested a deleterious effect.

### Room Temperature Magnet Placement

To control for possible crush injury created from pressure of the magnets used to induce frostbite, the procedure was repeated in a group of mice that received no frostbite injury but had room temperature magnets (21° C.) applied on their dorsal skin (crush control group). Post-procedure analgesia included administration of meloxicam at 1 mg/kg/day for 3 days. Another group of mice that received no frostbite injury or room temperature magnet placement served as healthy controls. All groups of mice were sacrificed by CO₂ euthanasia on day 15 after frostbite induction or room temperature magnet placement, and samples of the injured dorsal skin tissue were collected and stored in 10% neutral buffered formalin for histopathological analysis. The animals were euthanized on day 15 because this is the time point at which markers of wound healing were expected to be best seen in the wound as previously reported [5].

### Histopathology and Immunofluorescence Staining

The formalin-fixed skin tissue samples were embedded in paraffin blocks and sectioned at 8-μm thick on a microtome and collected on SuperFrost Plus® glass slides (J1800AMNZ, Thermo Fisher Scientific Inc., Germany). Histology was performed on the tissue sections using hematoxylin and eosin (H&E) staining to visualize general tissue morphology and assess the degree of tissue injury. A modified version of a previously developed histological scoring system established by Abramov et al [6] was used for this study. The histopathological scoring was assessed blindly by a dermatopathologist for 5 parameters of healing (acute inflammation, chronic inflammation, amount of granulation tissue, fibroblast maturation and re-epithelialization) and assigned a score as per the following scheme: 0=None; 1=Scant; 2=Moderate; 3=Abundant. Acute inflammation was scored based on the presence of neutrophils while chronic inflammation was evaluated based on the presence of plasma cells and monocytes [6]. Fibroblast maturation was determined on the basis of shape and alignment of these cells [6].

Immunofluorescence staining was performed to detect the expression of cleaved caspase-3 (1:100; a marker for apoptosis and is being used to assess frostbite damage), CD31 (1:100; a marker of vascularization and angiogenesis, as it is found on early endothelial cells), KI-67 (1:50; a marker of proliferation, which is abundant in a healing wound as granulation tissue forms and re-epithelialization occurs), fibronectin (1:100; secreted by fibroblasts to generate granulation tissue, which is the new connective tissue that covers a wound.), cytokeratin (1:200; a marker of differentiation and proliferation of keratinocytes, which are important for re-epithelialization of a wound) and CD163 (1:200; a macrophage-specific marker during wound healing) [7, 8]. These antibodies were purchased from Abcam in Toronto, Canada to examine the degree of frostbite injury as well as important healing stages including angiogenesis, re-epithelialization, granulation tissue formation and inflammation. Following a rehydration step in xylene and decreasing series of alcohol (100%, 95%, 70% and 50%) and de-ionized water, the tissue sections were washed with 1× phosphate buffered saline (PBS) for 10 minutes. The sections were subjected to antigen retrieval with citrate buffer (pH=6.0) at 95° C. for 10 minutes after which they were washed with 1×PBS for 5 minutes and incubated in a blocking buffer (1% horse serum in PBS) for 30 minutes at room temperature to block any non-specific staining between primary antibodies and the tissue The primary antibodies were diluted in 1% bovine serum albumin (BSA) and 200 μL of the antibody dilution was added to each tissue slide and incubated overnight at 4° C. to allow for optimal specific binding of the antibodies to tissue targets and reduce non-specific background staining. Following a washing step (3 times with 1×PBS) for 15 minutes, the tissue sections were incubated in the respective secondary antibodies (diluted in 1% BSA; 200 μL per tissue section) for 60 minutes at room temperature after which the tissue sections were washed again 3 times (with 1×PBS) for 15 minutes. To detect nuclei signals, 300 μL of the diluted solution of 40,6-diamidino-2-phenylindole (DAPI, 1 μL/mL; D9542, Sigma-Aldrich, Oakville, Canada) was added to each tissue section and incubated in the dark for 2 minutes at room temperature. DAPI binds to DNA and is a convenient nuclear counterstain. It has an absorption maximum at 358 nm and fluoresces blue at an emission maximum of 461 nm. The sections were then rinsed once with PBS and mounted with an anti-fade mounting media. The stained tissue sections were visualized and relative fluorescence images of the sections were taken with a wide-field fluorescent microscope (Olympus IX83). Stained pixel areas of the tissues were quantified using ImageJ version 1.52 software (National Institute of Health, USA) and the results were expressed as arbitrary fluorescence unit.

### Statistical Analysis

Values are presented as mean±standard deviation (SD). Statistical analysis of all data was performed using GraphPad Prism software version 9 (La Jolla, CA). Data were analyzed using one-way analysis of variance (ANOVA) followed by Tukey's post-hoc test. Significant difference between groups was indicated with p<0.05.

### Results

Ap39 Hastened Healing of Frostbite Wound from Inflammatory to Proliferative Phase

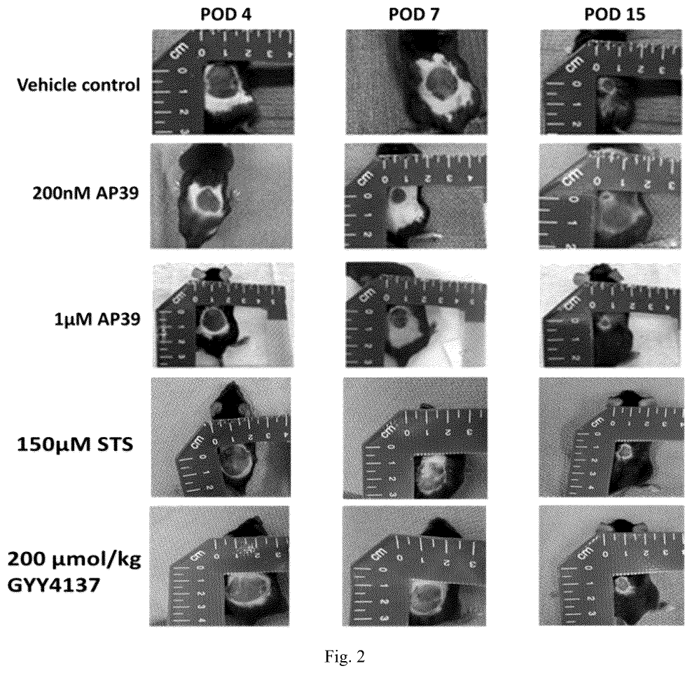

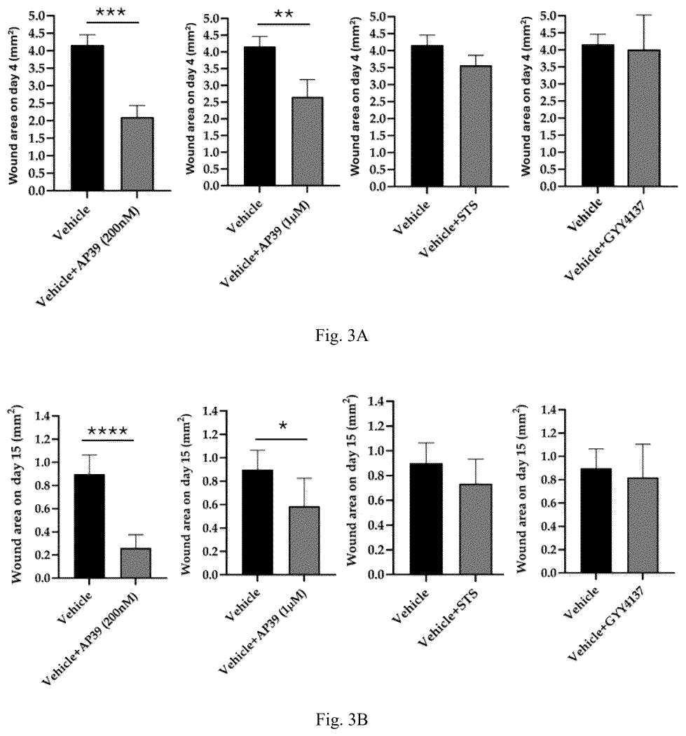

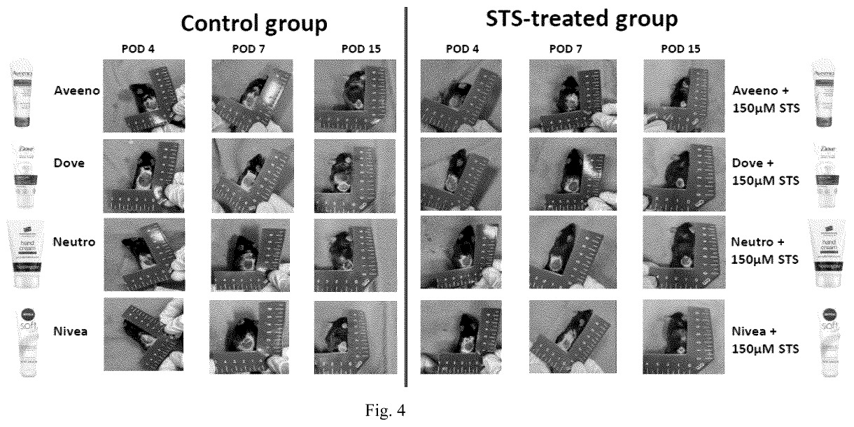

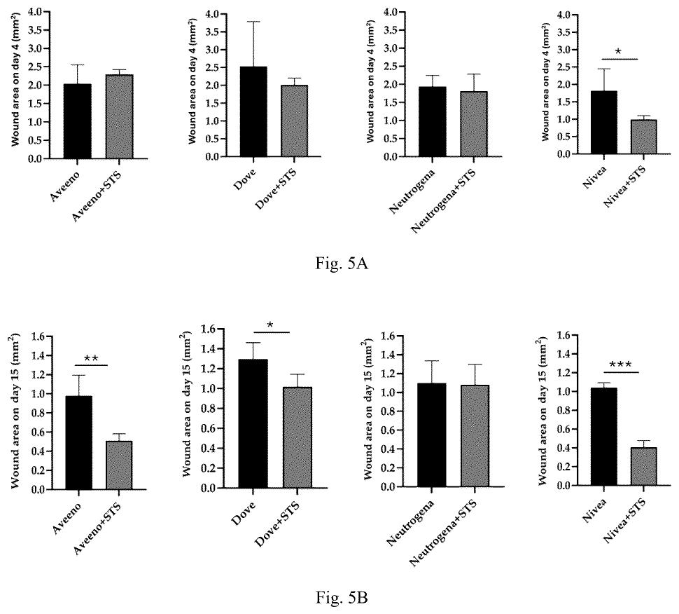

As shown in FIGS. 2, 3A and 3B, topical application of both concentrations of AP39 (200 nM and 1 μM) in the vehicle cream containing hyaluronan and phosphatidylethanolamine hastened frostbite wound healing as evidenced by progressively reduced wound size compared to vehicle-treated control mice. For topical application of STS and GYY4137 reduction of frostbite wound from day 4 to day 15 are slightly smaller than that of the vehicle control.

FIGS. 4, 5A and 5B compare the effect on frostbite wound healing using four different commercially available creams/moisturizers as vehicle controls to the effects of the topical application of STS with those four different commercially available creams/moisturizers. As shown in FIGS. 4, 5A and 5B STS in the vehicle control progressively reduced wound size compared to the corresponding vehicle control without STS. Quantification of the wound size shows that STS with Nivea® reduced wound size since at least day 4 (see FIG. 5A). STS with Aveeno®, Dove® and Nivea® have reduced wound size on day 15 compared to their respective vehicle controls.

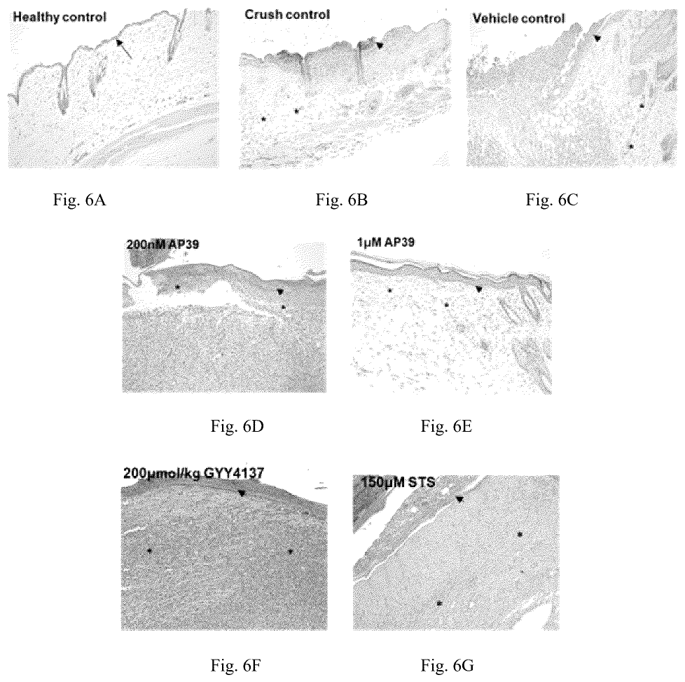

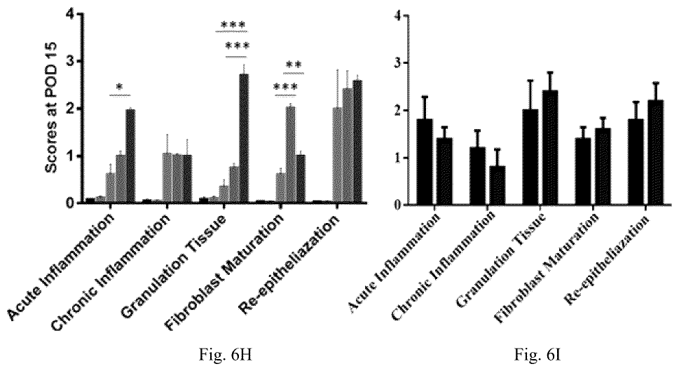

FIGS. 6A to 6G show histopathological changes on day 15 following induction of frostbite injury and treatment with and without the 3 different H₂S donors as well as injury from room temperature magnet placement (crush control). Normal skin shows intact epidermal layer (black arrow; FIG. 6A). Compared to healthy control skin (FIG. 6A), induction of frostbite injury caused regenerative changes with loss of epidermal layer (arrow heads), increased underlying dermal cellular infiltrates (*) and re-epithelialization, while placement of room temperature magnet resulted in thickening of the epidermal layer (FIGS. 6B to 6G). Quantitatively, acute inflammation was significantly greater in the 1 μM AP39-treated group than in the 200 nM AP39 and vehicle control groups (FIG. 6H; p<0.05) while chronic inflammation did not change in all three frostbite groups (FIG. 6H; p>0.05). Also, 1 μM concentration of AP39 markedly increased the amount of granulation tissue compared to that in 200 nM AP39 and vehicle control groups (FIG. 6H; p<0.05). However, fibroblast maturation was highest in 200 nM AP39-treated group in comparison with 1 μM AP39 and vehicle control groups (FIG. 6H; p<0.05). While AP39 strongly influenced acute inflammation, amount of granulation tissue and fibroblast maturation, it also increased wound re-epithelialization (epithelial restitution) albeit not significantly different compared to vehicle control group (FIG. 6H; p>0.05). Collectively, AP39 treatment hastened frostbite wound healing from inflammatory to proliferative phase characterized by increased formation of granulation tissue, fibroblast maturation and re-epithelialization.

FIGS. 6H and 6I are quantification of acute and chronic inflammation, granulation tissue formation, fibroblast maturation and re-epithelialization following induction of frostbite injury and treatment with H₂S donors (AP39, GYY4137 and STS). Quantitatively, acute inflammation was significantly greater in the 1 μM AP39-treated group than in the 200 nM AP39 and vehicle control groups (FIG. 6H; p<0.05) while chronic inflammation did not change in all three frostbite groups (FIG. 6H; p>0.05). Also, 1 μM concentration of AP39 markedly increased the amount of granulation tissue compared to that in 200 nM AP39 and vehicle control groups (FIG. 6H; p<0.05). However, fibroblast maturation was highest in 200 nM AP39-treated group in comparison with 1 μM AP39 and vehicle control groups (FIG. 6H; p<0.05). While AP39 strongly influenced acute inflammation, amount of granulation tissue and fibroblast maturation, it also increased wound re-epithelialization (epithelial restitution), albeit not significantly different compared to vehicle control group (FIG. 6H; p>0.05). Also, there was significant difference in all parameters in GYY4137- and STS-treated groups compared to vehicle control group (FIG. 6I; p<0.05), except in chronic inflammation, which did not show significant difference (FIG. 6I; p>0.05). Collectively, AP39, GYY4137 and STS treatment hastened frostbite wound healing from inflammatory to proliferative phase characterized by increased formation of granulation tissue, fibroblast maturation and re-epithelialization.

### Ap39 Promoted Angiogenesis, Proliferation and Granulation Tissue Formation

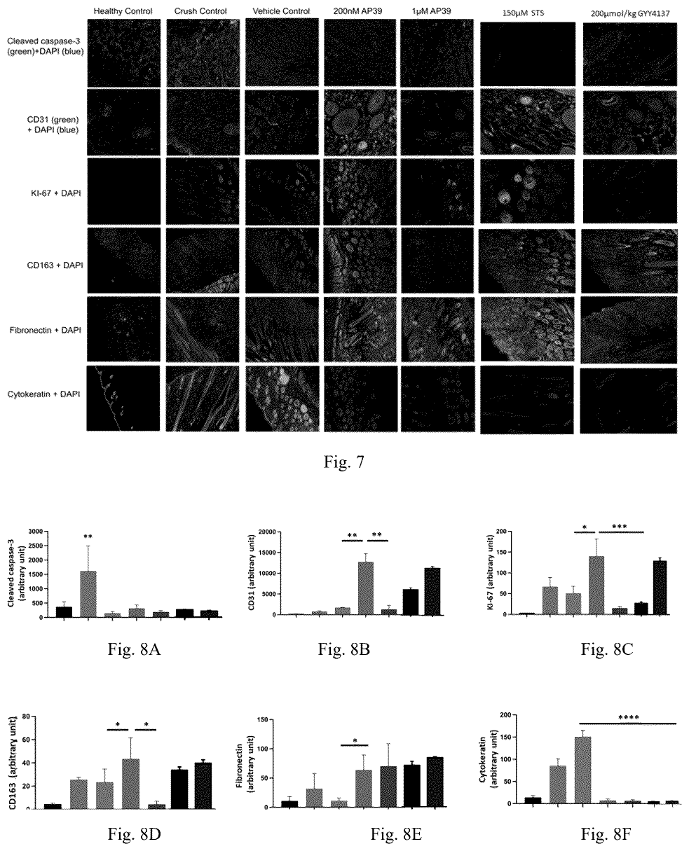

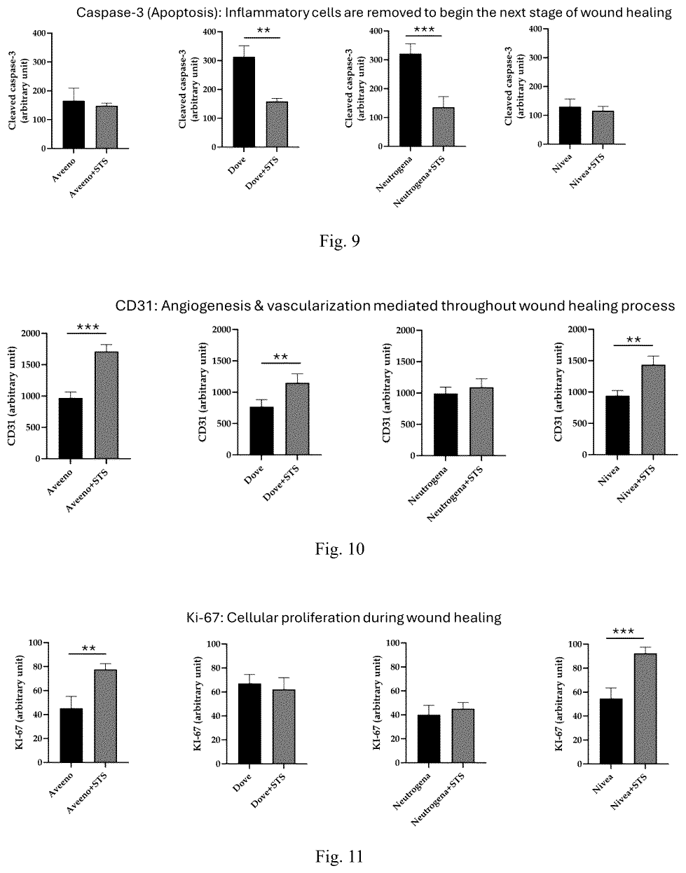

We determined the expression of markers of apoptosis (cleaved caspase-3), vascularization and angiogenesis (CD31) and cell proliferation (KI-67) following induction of frostbite injury and injury from room temperature magnet placement. Cleaved caspase-3 was abundantly expressed in the wound of mice that received room temperature magnet placement while its expression was surprisingly and significantly reduced in all groups including vehicle control group to the same level with both AP39-treated groups (FIGS. 7 and 8A; p<0.01). Thus, AP39, STS and GYY4137 had no effect on apoptosis in frostbite injury. However, CD31 and KI-67 proteins were markedly expressed in the wounds of 200 nM AP39-treated mice while their expressions were significantly reduced in the wounds of all other groups including 1 μM AP39-treated and vehicle control groups (FIGS. 75, 8B and 8C; p<0.05). In addition to these markers, we also evaluated the expression of CD163 (M2-like anti-inflammatory macrophages), fibronectin (granulation tissue formation) and cytokeratin (epidermal keratinocyte hyperproliferation). While CD163 expression (FIGS. 7 and 8D) followed the same pattern as observed in FIGS. 5 and 8B-8C, suggesting increased anti-inflammatory activity in the wounds of 200 nM AP39-treated mice, fibronectin was equally expressed in both AP39 groups, with significantly increased expression compared to vehicle control mice (FIGS. 7 and 8E). Finally, cytokeratin expression in all H₂S donor-treated groups significantly decreased while its expression markedly increased in vehicle control mice (FIGS. 7 and 8F; p<0.0001). Taken together, topical application of H₂S donor promoted angiogenesis, cell proliferation and generation of granulation tissue, leading to quick healing of frostbite wound.

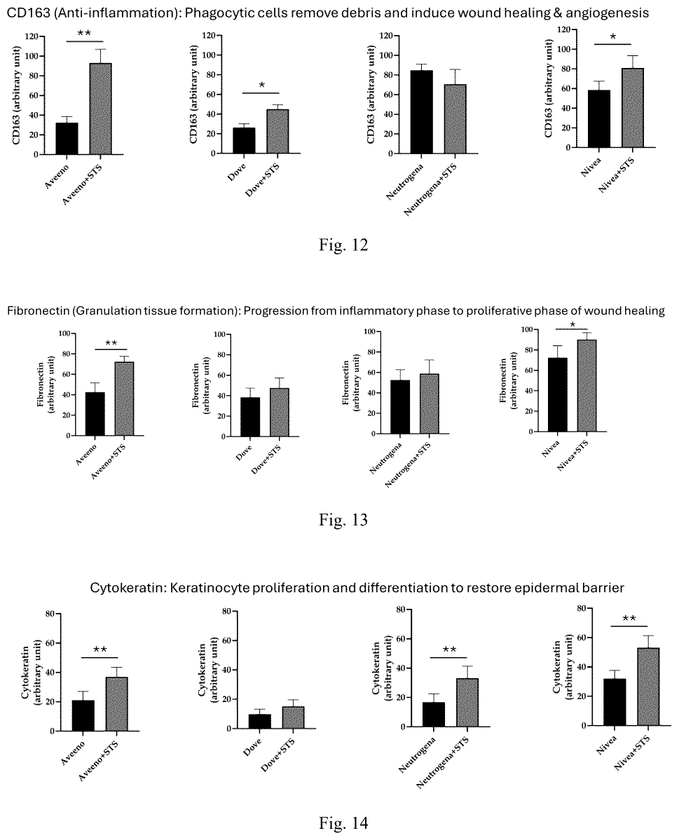

Treatment with STS increased fibroblast proliferation (FIG. 11), vascularization (angiogenesis) (FIGS. 10 and 12), tissue granulation (FIG. 13) and keratinocyte proliferation and differentiation (FIG. 14).

As shown in FIG. 10, addition of STS to Aveeno, Dove and Nivea creams resulted in significant upregulation of CD31 expression compared to treatments with these creams but without STS application. This implies that STS mediates angiogenesis and vascularization throughout wound healing process. No significant change in CD31 expression was observed following Neutrogena supplementation with 150 μM of STS.

FIG. 11 illustrates that supplementation of Aveeno and Nivea creams with 150 μM of STS markedly upregulated KI-67 expression relative to treatments with Aveeno and Nivea but without STS application, suggesting that STS favors cellular proliferation during wound healing Addition of the same dose of STS to Dove and Neutrogena creams did not change KI-67 expression significantly.

Addition of 150 μM of STS to Aveeno, Dove and Nivea creams caused significant increase in CD163 expression compared to treatments with Aveeno, Dove and Nivea without STS application. Such a significant increase means that at a dose of 150 μM, STS activates anti-inflammation process, including removal of debris by phagocytic cells remove to induce wound healing and angiogenesis (see FIG. 12). Addition of the same dose of STS to Neutrogena group caused a decrease in CD163 expression albeit not significant (see FIG. 12).

With reference to FIG. 13, supplementation of Aveeno and Nivea creams with 150 μM of STS significantly increased fibronectin expression in comparison with treatments with Aveeno and Nivea without STS application. This increase implies that STS promotes granulation tissue formation, which enhances progression from inflammatory phase to proliferative phase of wound healing. Supplementation of Dove and Neutrogena creams with the same dose of STS did not increase fibronectin expression significantly.

FIG. 14 shows that STS at a dose of 150 μM significantly increased cytokeratin expression in Aveeno, Neutrogena and Nivea groups compared to treatments with these creams but without STS application. This suggests that STS promotes keratinocyte proliferation and differentiation to restore epidermal barrier. No significant change was observed in cytokeratin in Dove group upon addition of the same dose of STS.

The present study investigated whether hydrogen sulfide (H₂S), through the use of donor compounds (AP39, GYY4137 and sodium thiosulfate [STS]), applied topically in a vehicle cream could mitigate frostbite injury and improve wound healing in mice. Frostbite injury was induced continuously for 3 minutes on the dorsal skin of C57BL/6 mice (Mus musculus) using magnets frozen on dry ice (−80° C.). H₂S donor compounds, delivered via a vehicle cream, were used daily to treat frostbite injury until animals were euthanized on day 15 after induction of frostbite injury. Wound tissues were stained with hematoxylin and eosin along with immunofluorescence staining with cleaved caspase-3, CD31, KI-67, CD163, fibronectin and cytokeratin. All three H₂S donor compounds improved granulation tissue maturation (p<0.001), angiogenesis (p<0.01) and cell proliferation (p<0.001) compared to vehicle control. Thus, H₂S donor compounds improved frostbite wound healing, and therefore H₂S and H₂S donor compounds can be used in the treatment of frostbite injury in a patient.

Treatment of frostbite wounds with AP39, GYY4137 and STS improved wound healing compared to vehicle control group. This was characterized by improved granulation tissue formation (p<0.001), angiogenesis (p<0.01) and cell proliferation (p<0.001) compared to vehicle control. The result also showed that administration of STS via Aveeno® and Nivea® creams improved wound healing compared to Dove® and Neutrogena®. In conclusion, H₂S donor molecules (AP39, GYY4137 and STS) improved frostbite wound healing, and therefore H₂S donor molecules are useful in the treatment of patients with frostbite injury, including acute frostbite injury.

### REFERENCES

1. Auerbach L J, Galvez M G, De Clerck B K, Glotzbach J, Wehner M R, Chang E I, Gurtner G C, Auerbach P S. A novel mouse model for frostbite injury. Wilderness Environ Med. 2013; 24(2):94-104.
2. Symonette C J, Kaur Mann A, Tan X C, Tolg C, Ma J, Perera F, Yazdani A, Turley E A. Hyaluronan-phosphatidylethanolamine polymers form pericellular coats on keratinocytes and promote basal keratinocyte proliferation. Biomed Res Int. 2014; 2014:727459.
3. Le Trionnaire S, Perry A, Szczesny B, et al. The synthesis and functional evaluation of a mitochondria-targeted hydrogen sulfide donor, (10-oxo-10-(4-(3-thioxo-3H-1,2-dithiol-5-yl)phenoxy)decyl)triphenylphosphonium bromide (AP39). Med Chem Commun 2014; 5:728-736.
4. Lobb I, Jiang J, Lian D, Liu W, Haig A, Saha M N, Torregrossa R, Wood M E, Whiteman M, Sener A. Hydrogen Sulfide Protects Renal Grafts Against Prolonged Cold Ischemia-Reperfusion Injury via Specific Mitochondrial Actions. Am J Transplant. 2017; 17(2):341-352.
5. Zhao X, Liu L, An T, Xian M, Luckanagul J A, Su Z, Lin Y, Wang Q. A hydrogen sulfide-releasing alginate dressing for effective wound healing. Acta Biomater. 2020; 104:85-94.
6. Abramov Y, Golden B, Sullivan M, Botros S M, Miller J J, Alshahrour A, Goldberg R P, Sand P K. Histologic characterization of vaginal vs. abdominal surgical wound healing in a rabbit model. Wound Repair Regen. 2007; 15(1):80-6.
7. Andreoli A, Ruf M T, Sopoh G E, Schmid P, Pluschke G. Immunohistochemical monitoring of wound healing in antibiotic treated Buruli ulcer patients. PLOS Negl Trop Dis. 2014; 8(4):e2809.
8. Wang G G, Li W. Hydrogen sulfide improves vessel formation of the ischemic adductor muscle and wound healing in diabetic db/db mice. Iran J Basic Med Sci. 2019; 22(10):1192-1197.

Although various embodiments of the disclosure have been described and illustrated, it will be apparent to those skilled in the art in light of the present description that numerous modifications and variations can be made. The scope of the invention is defined more particularly in the appended claims. All publications referenced in this disclosure are incorporated herein by reference.

What is claimed is:

1. A method of treating frostbite injury in a patient comprising administering to the patient H₂S.

2. The method of claim 1, wherein said administering comprises topically contacting the frostbite injury with the H₂S.

3. The method of claim 1, wherein the H₂S is provided as an H₂S donor.

4. The method of claim 1, wherein the H₂S is provided in a composition comprising the H₂S and a vehicle.

5. The method of claim 4, wherein the vehicle contains hyaluronic acid.

6. The method of claim 4, wherein the vehicle contains phosphatidylethanolamine.

7. The method of claim 5, wherein the vehicle contains phosphatidylethanolamine.

8. The method of claim 4, wherein the vehicle is a water-in-oil emulsion.

9. The method of claim 4, wherein the vehicle is an oil-in-water emulsion.

10. The method of claim 4, wherein the vehicle is a commercially available skin moisturizer.

11. The method of claim 1, wherein the H₂S donor is selected from STS, AP39 and GYY4137.

12. A method of preventing frostbite injury in a subject comprising administering to the subject H₂S.

13. A composition comprising H₂S and a skin moisturizer.

14. The composition of claim 13, wherein the H₂S is provided as a H₂S donor.

15. The composition of claim 14, wherein the H₂S donor is selected from STS, AP39 and GYY4137.

16. The composition of claim 13, wherein the skin moisturizer comprises hyaluronic acid and phosphatidylethanolamine.

17. The composition of claim 14, wherein the skin moisturizer comprises hyaluronic acid or phosphatidylethanolamine.

18. The composition of claim 14, wherein the skin moisturizer is a commercially available skin moisturizer.
